(19) United States
(12) Patent Application Publication
(10) Pub. No.: US 2009/0215176 A1
(43) Pub. Date: Aug. 27, 2009

(54) Differential Pressure Pump System

(75) Inventor: David E. Orr, Lafayette, IN (US)

Correspondence Address: DORITY & MANNING, P.A., POST OFFICE BOX 1449, GREENVILLE, SC, 29602-1449, US

(73) Assignee: CLEMSON UNIVERSITY, Clemson, SC (US)

(21) Appl. No.: 12/392,146

(22) Filed: Feb. 25, 2009

Related U.S. Application Data
(60) Provisional application No. 61/031,088, filed on Feb. 25, 2008.

(51) Int. Cl. C12N 5/06; C12M 1/00

(52) U.S. Cl. 435/383; 435/294.1

(57) ABSTRACT

The present disclosure is directed to a fluid pumping mechanism that uses differential pressure to drive flow through one or more culture chambers. Fluid contained within a first chamber can be caused to flow through one or more culture chambers and thence to a second chamber upon establishment of a pressure differential between the two chambers. The differential pressure system can induce either steady state or pulsatile flow through a culture chamber. In one embodiment, a culture chamber can be held at a high or low pressure hydrostatic state through utilization of the disclosed systems.

### CROSS REFERENCE TO RELATED APPLICATIONS

This application claims filing benefit of U.S. Provisional Patent Application Ser. No. 61/031,088 having a filing date of Feb. 25, 2008, which is incorporated herein by reference in its entirety.

### BACKGROUND

The ability to culture in vitro viable three-dimensional cellular constructs that mimic natural tissue has proven very challenging. The ideal in vitro system should accurately model the essential cellular interactions found during in vivo development under in vitro conditions that will not bring harm to the cells or the various biochemical agents involved in the working system. For example, it is commonly desired that the conditions for successful growth and development of a tissue construct mimic biological conditions as closely as possible with regard to temperature, pH, mechanical and chemical stimuli, and the like. Successful growth conditions must also provide a means for communication of agents such as nutrients, growth factors, and the like, to the developing cellular constructs as well as a means for removing waste materials from the developing constructs. Hence, dynamic culture systems generally include one or more mechanical pumping apparatuses to provide a fluid flow over and/or through the developing constructs.

Unfortunately, mechanical pumping systems have been found problematic in tissue culture. For instance, fluid flow characteristics can be difficult to maintain within the desired parameters. Mechanical pumping systems can include periodic flow disruption as well as sudden spiking and drops of flow velocity. Such inconsistency of the flow can cause high shear rates leading to physical and developmental damage to tissue constructs, individual cells, as well as active agents (e.g., proteins, nutrients, and the like) that can be contained in the fluid being pumped as well as in the culture.

A need currently exists in the art for an in vitro cell culture system that can include fluid flow control devices and methods that will not lead to damage of materials contained in a pumped fluid culture medium and also will not damage components, e.g., cells, of the developing culture. What is also needed in the art is a dynamic cell culture system including a means of providing safe, effective and constant fluid flow through the developing culture.

### SUMMARY

In one embodiment, the present disclosure is directed to a biological culture system including a fluid pumping control mechanism based upon differential pressure to drive flow through a culture chamber. For example, a system can include a first pressure chamber, a culture chamber located downstream of the first pressure chamber and a fluid receptacle located downstream of the culture chamber. The biological culture system also includes a differential pressure control system that controls fluid flow from the first pressure chamber and through the culture chamber via the establishment of a differential pressure gradient across the system.

The system can be a co-culture system. For instance, the system can include two, three, or even more separate culture chambers in biochemical communication with one another. The separate culture chambers of a co-culture system can include a shared fluid flow control system or can have separate fluid flow control systems.

During use, flow to the culture chamber can be controlled according to the pressure gradient. For instance, flow to the culture chamber can be steady-state flow or can be pulsatile, as desired. The system can also be controlled so as to hold the culture chamber at hydrostatic compression.

A culture chamber can contain any desired biological specimen. For instance, in one embodiment, a culture chamber can contain one or more types of living cells. For example, a culture chamber can contain an engineered tissue culture.

### BRIEF DESCRIPTION OF THE FIGURES

A full and enabling disclosure, including the best mode thereof, to one of ordinary skill in the art, is set forth more particularly in the remainder of the specification, including reference to the accompanying figures, in which:

FIG. 1 is a schematic diagram of a differential pressure pump system such as may be used in one embodiment of the present disclosure; and

FIG. 2 is a schematic diagram of one embodiment of a co-culture bioreactor as may be utilized in conjunction with a differential pressure pump system as disclosed herein.

Repeat use of reference characters in the present specification and drawings is intended to represent the same or analogous features or elements of the present disclosure.

### DETAILED DESCRIPTION

Reference will now be made in detail to various embodiments of the disclosed subject matter. Each embodiment is provided by way of explanation of the disclosure, not limitation of the disclosure. In fact, it will be apparent to those skilled in the art that various modifications and variations may be made in the present disclosure without departing from the scope or spirit of the disclosure. For instance, features illustrated or described as part of one embodiment, may be used in another embodiment to yield a still further embodiment. Thus, it is intended that the present disclosure cover such modifications and variations as come within the scope of the appended claims and their equivalents.

The present disclosure is generally directed to systems that can be used to transfer fluid to, across and/or through a biological culture. Disclosed systems can be advantageous over previously known mechanical pumping systems such as those incorporating peristaltic pumps. For instance, disclosed systems can minimize feedback effects during the commencement and halting of fluid flow and can thus prevent damage to components of a cell culture held within the flow field. Disclosed systems can also provide excellent control of flow characteristics, particular in low flow velocity embodiments.

One embodiment of a system as disclosed herein can include two chambers and can be activated to impose a pressure gradient between the two chambers. In addition, at least the first of the chambers can be structured so as to contain a fluid. One or more culture modules can be located between the two chambers and upon establishment of a pressure gradient, fluid can flow from the first chamber to the culture module(s) and, optionally, on to a second chamber. The fluid can thus be utilized to carry, by way of example, nutrients, antibiotics, growth factors, and the like to a developing culture contained in a culture module as well as to carry waste products away from the culture.

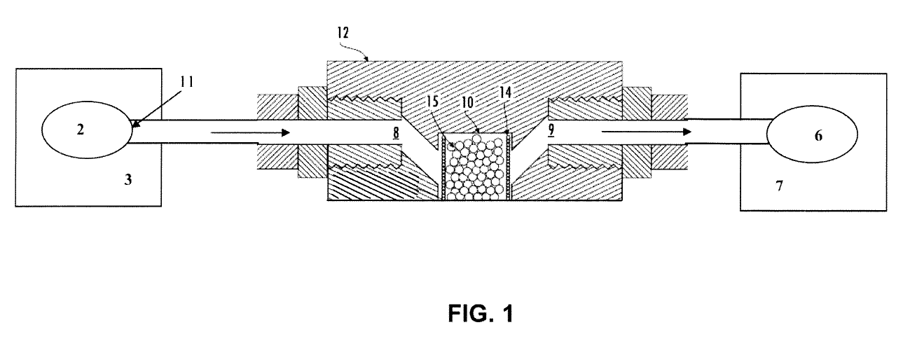

FIG. 1 depicts one embodiment of a differential pressure pumping system as may be utilized with a culture system as disclosed herein. As can be seen, the system includes a first pressure chamber 3. The interior of pressure chamber 3 can be isolated from the surrounding atmosphere utilizing any suitable methods and materials. For instance, pressure chamber 3 can be formed of a material such as glass, plastic, metal, or the like that can adequately withstand the operational pressures to be expected within the pressure chamber 3, e.g., between about 0 and about 500 kPa. Higher operational pressures can be expected in other embodiments, for instance operational pressures up to about 10 MPa, in one embodiment. In one embodiment, pressure chamber 3 can also include an access port (not shown) so as to replenish fluids located within the pressure chamber 3.

Pressure chamber 3 can be connected to a high pressure gas source, such as an air, oxygen, carbon dioxide, or nitrogen source via lines, valves, regulators and the like, as are generally known in the art. Alternatively, pressure chamber 3 can be directly connected to a compressor that can deliver compressed air directly into pressure chamber 3.

Pressure chamber 3 can be designed so as to contain a fluid. For instance, in the embodiment illustrated in FIG. 1, pressure chamber 3 can hold a container 2 that can be separable from the interior of pressure chamber 3 and can contain a fluid. Containment of a fluid within pressure chamber 3 via a separable container 2 may be preferred in some embodiments as this can simplify replacement of fluid within chamber 3 and cleaning of chamber 3. Use of separable container 2 is not a requirement of the disclosed systems, however, and in other embodiments a fluid can be directly located within pressure chamber 3.

When utilized, container 2 can generally be formed of a pliable material. For instance, container 2 can be a pliable sack or bag formed of a flexible polymeric material such as poly(vinyl chloride), silicone and the like. In one embodiment a container 2 can be gas-permeable. According to such an embodiment, the gaseous contents of the pressure chamber can affect the make up of the fluid to be pumped during a process. For instance, atmospheric gas supplemented with 5% carbon dioxide can be utilized to pressurize the chamber 3, and a fluid held within a gas permeable container 2 can thus be oxygenated so as to deliver an appropriate dissolved oxygen content to a developing cell culture.

Referring again to FIG. 1, container 2 includes an outlet 11 that connects container 2 to line 8. Outlet 11 and/ or line 8 can optionally include one or more control valves (not shown) that can be used to isolate a culture chamber, for instance during replacement or re-filling of container 2. In general, an outlet 11 can be opened or shut when the differential pressure across the culture chamber 10 is at a minimum followed by the gradual development of the pressure differential such that flow through the culture chamber 10 is initiated (or stopped) by the gradual change in the pressure differential rather than a sudden change due to the opening or closing of a valve.

As can be seen, line 8 can connect container 2 to culture module 12 that can contain, for instance, a tissue culture. In this particular embodiment, the illustrated culture system includes a single culture chamber 10 that is defined by a culture module 12, though in other embodiments, described in further detail below, a culture system can include multiple culture chambers. The dimensions and overall size of a culture module 12, and culture chamber 10, are not critical to the disclosed systems. In general, a culture module 12 can be of a size so as to be handled and manipulated as desired, and so as to provide access to the culture chambers therewithin either through disassembly of the device, through a suitably located access port, or according to any other suitable method. In addition, a culture chamber 10 defined by the module 12 can generally be of any size, for instance of a size so as to cultivate living cells within and to ensure adequate nutrient flow throughout a three-dimensional cellular construct growing in the culture chamber 10 and prevent cell death at the construct center due to lack of nutrient supply.

In general, a module 12 can be formed of any moldable or otherwise formable material. The surface of the culture chamber 10, as well as any other surfaces of the module that may come into contact with cells, nutrients, growth factors, or any other fluids or biochemicals that may contact the cells, can be of a suitable sterilizable, biocompatible material. In one particular embodiment, components of the system can also be formed so as to isolate cell attachment to a porous biomaterial matrix structure and discourage cell anchorage to surfaces of culture chamber 10.

Culture chamber 10, can be in fluid communication with container 2 via line 8 and can generally be of a shape and size so as to cultivate living cells within the chamber 10. In one preferred embodiment, culture chamber 10 can be designed to accommodate a biomaterial scaffold within the culture chamber 10. For instance, a culture chamber 10 can be between about 3 mm and about 10 mm in a cross sectional dimension. In another embodiment, a culture chamber can be greater than about 5 mm in every cross sectional direction. For instance, a chamber 10 can be cylindrical in shape and about 5-10 mm in cross sectional diameter and height. It should be understood, however, that the shape of culture chamber 10 is not critical to the disclosed subject matter.

A system can include a cell construct that can be contained in a culture chamber 10. The term “cell construct” as utilized herein refers to one or more articles upon which cells can attach and develop. For instance, the term “cell construct” can refer to a single continuous scaffold, multiple discrete scaffolds, or a combination thereof. The terms “cell construct,” “cellular construct,” “construct,” and “scaffold” are intended to be synonymous. Any suitable cell construct as is generally known in the art can be located in a culture chamber 10 and can provide anchorage sites for cells and to encourage cellular growth and development within the culture chamber 10. In addition, generally any cell type can be cultured according to disclosed methods and devices. For instance, cell types from any species can be cultured. By way of example, human cell types as may be cultured as described herein can include, without limitation, adult stem cells, cancer, normal tissue, biopsy tissue, cell lines, etc. In one preferred embodiment, cell types that can exhibit increased physiologic relevance when cultured in three dimensions as compared to the same cell types when cultured in two dimensions can be cultured according to disclosed methods and devices. For instance, hepatocytes and many cancer cells can exhibit increased physiologic relevance when cultured in three dimensions.

For purposes of the present disclosure, the term continuous scaffold generally refers to a material suitable for use as a cellular construct that can be utilized alone as a single, three-dimensional entity. A continuous scaffold is usually porous in nature and has a semi-fixed shape. Continuous scaffolds are well known in the art and can be formed of many materials, e.g., coral, collagen, calcium phosphates, synthetic polymers, decellularized protein matrices and the like, and are usually pre-formed to a specific shape designed for the location in which they will be placed. Continuous scaffolds are usually seeded with the desired cells through absorption and cellular migration, often coupled with application of pressure through simple stirring, pulsatile perfusion methods or application of centrifugal force.

Discrete scaffolds are smaller entities, such as beads, rods, tubes, fragments, or the like. When utilized as a cellular construct, a plurality of identical or a mixture of different discrete scaffolds can be loaded with cells and/or other agents and located within a void where the plurality of entities can function with composite engineered properties for a desired cellular response. Exemplary discrete scaffolds suitable for use in disclosed systems are described further in U.S. Pat. No. 6,991,652 to Burg, which is incorporated herein by reference. A cellular construct formed of a plurality of discrete scaffolds can be preferred in certain embodiments as discrete scaffolds can facilitate uniform cell distribution throughout the construct and can also allow good flow characteristics throughout the porous construct as well as encouraging the development of a viable three-dimensional cell culture.

In one embodiment, for instance when considering a cellular construct including multiple discrete scaffolds, the construct can be seeded with cells following assembly and sterilization of the system. For example, a construct including multiple discrete scaffolds can be seeded in one operation or several sequential operations. Optionally, the construct can be pre-seeded, prior to assembly of the system. In one embodiment, the construct can include a combination of both pre-seeded discrete scaffolds and discrete scaffolds that have not been seeded with cells prior to assembly of the system.

Construct materials can generally include any suitable biocompatible material. For instance, a cellular construct can include biodegradable synthetic polymeric scaffold materials such as, without limitation, polylactide, chondroitin sulfate (a proteoglycan component), polyesters, polyethylene glycols, polycarbonates, polyvinyl alcohols, polyacrylamides, polyamides, polyacrylates, polyesters, polyetheresters, polymethacrylates, polyurethanes, polycaprolactone, polyphophazenes, polyorthoesters, polyglycolide, copolymers of lysine and lactic acid, copolymers of lysine-RGD and lactic acid, and the like, and copolymers of the same. Optionally, a construct can include naturally derived biodegradable materials including, but not limited to chitosan, agarose, alginate, collagen, hyaluronic acid, and carrageenan (a carboxylated seaweed polysaccharide), demineralized bone matrix, and the like, and copolymers of the same.

A biodegradable construct material can include factors that can be released as the scaffold(s) degrade. For example, a construct can include within or on a scaffold one or more factors that can trigger cellular events. For instance, as the scaffold(s) forming the cellular construct degrades, the factors can be released to interact with the cells.

Referring again to FIG. 1, in those embodiments including a cellular construct formed with a plurality of discrete scaffolds, a retaining mesh 14 can also be located within the culture chamber 10. The retaining mesh 14 can be formed of any suitable biocompatible material, such as polypropylene, for example, and can line at least a portion of a culture chamber 10, so as to prevent material loss during media perfusion of the culture chamber 10. A porous retaining mesh 14 can generally have a porosity of a size so as to prevent the loss of individual discrete scaffolds within the culture chamber 10. For example, a retaining mesh 14 can have an average pore size of between about 100 μm and about 150 μm.

On the downstream side of culture module 12 is an outlet line 9 that connects culture chamber 10 to a second container 6 held within a second chamber 7 such that culture chamber 10 can be in fluid communication with container 6. Second container 6 can, in one embodiment, be of similar construction as first container 2, though this is not a requirement of the presently disclosed system. Moreover, as with the upstream side of the system, the inclusion of second container 6 in the differential pressure flow system is not a requirement, and in one embodiment fluid carried in line 9 can empty directly into chamber 7. Chamber 7 can be held at atmospheric conditions or can be capable of being pressurized, as discussed further below.

During use, pressure within chamber 3 can be increased and the pressure differential can force fluid out of container 2 and through line 8. The fluid can carry beneficial material to culture chamber 10. For instance, a fluidic nutritive additive can be delivered to a culture within culture chamber 10.

Chamber 6 can be at atmospheric pressure or can be at increased pressure, as desired. For instance, chamber 6 can be held at an increased pressure relative to the surrounding atmosphere but at a pressure slightly less than that of chamber 3 so as to provide improved control of flow characteristics between container 2 and container 6. Differential pressure control of the fluid flow through culture chamber 10 is particularly suitable for in vitro biomedical applications due at least in part to the minimal presence of mechanical influence or interference of flow. This is particularly beneficial when working with living cells and/or delicate agents such as proteins and growth factors common to cell culturing protocols.

The local environment within culture chamber 10 can be controlled, for instance to provide stable environmental conditions for a culture held in culture chamber 10. For example, pressure can be controlled within the first pressure chamber 3 and/or the second chamber 7, so as to provide a local environment within the culture chamber 10 at about atmospheric pressure. As the system can be isolated from the surrounding atmosphere, in other embodiments the pressure within culture chamber 10 can be above or below atmospheric, as desired. For instance, in one embodiment, pressures within the first pressure chamber 3 and the second chamber 7 can be elevated and equalized to create a hydrostatic compression environment within culture chamber 10. Optionally, pressure in both chambers 3, 7 can be elevated above atmospheric while maintaining a pressure differential between the two. Thus, culture chamber 10 can be at higher pressure (i.e., higher than surrounding atmospheric pressure) while flow is maintained through culture chamber 10. Similarly, both chambers 3, 7 can be held at a lower pressure, either the same or different as one another, so as to maintain culture chamber 10 at a vacuum pressure either with our without flow therethrough, as desired.

The disclosed systems can also be utilized to establish pulsatile flow through a culture chamber 10. For instance, pressure in one or both of chambers 3, 7 can fluctuate through the use of controlled pressure regulators and the like so as to provide a controlled pulsatile flow through culture chamber 10. Disclosed pulsatile flow systems can more closely mimic the natural pulsatile characteristics of fluid flow (e.g., blood, lymph, etc.) without damaging either biological components in the fluid or those held in culture chamber 10. More specifically, the use of disclosed differential pressure controlled flow systems is believed to lessen the possibility of damage to biological components due to sudden changes in flow characteristics common in previously known mechanically controlled systems.

Disclosed systems can also include components for control of other aspects of the local environment within culture chamber 10 such as temperature, gaseous content, and the like. For instance, the gaseous composition of the local atmosphere within culture chamber 10 can be monitored and controlled, for instance via gaseous content of chamber 3 combined with utilization of a gas permeable container 2, as discussed above. Control of other environmental characteristics, such as temperature, can be facilitated according to any suitable control system as is known in the art.

Flow from culture chamber 10 to container 6 via line 9 as indicated by the directional arrow in FIG. 1 can carry waste products generated within culture chamber 10. Flow can be stopped, for instance with a gradual lessening of the differential pressure across the system followed by the closing of valves (not shown) in lines 8, 9, for instance to refill, empty, or replace the containers 2, 6.

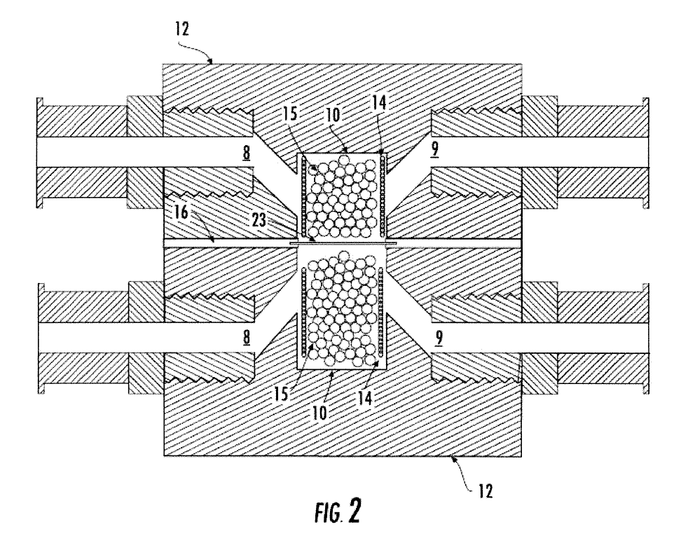

Disclosed systems are not limited to a single culture chamber. FIG. 2 illustrates a co-culture bioreactor system as may be utilized with a differential pressure system as disclosed herein. According to this embodiment, two culture chambers 10, 10 can be aligned so as to be immediately adjacent to one another. A co-culture system is not limited to two culture chambers, however, and multiple culture chambers can be combined according to the disclosed subject matter.

In the illustrated embodiment, a gasket 16 including a permeable membrane portion 23 can be located between culture chambers 10, 10. For instance, the membrane portion 23 located between the two culture chambers 10, 10 can have a porosity that can allow biochemical materials, for instance growth factors produced by a cell in one chamber, to pass through the membrane and into the adjacent chamber. Accordingly, biochemical communication can occur between the two chambers, for instance between cells contained in the first chamber and cells contained in the second chamber. The membrane porosity can generally be small enough to prevent passage of cells or cell extensions from one chamber to another. For instance, the membrane porosity can be predetermined so as to discourage physical contact between cells held in adjacent chambers, and thus maintain physical isolation of cell types, while allowing biochemical communication between cells held in separate chambers. Suitable porosity for a membrane can be determined based upon specific characteristics of the system, for instance the nature of the cells to be cultured within the chamber(s). Such determination is well within the ability of one of ordinary skill in the art and thus is not discussed at length herein.

Physical isolation of cellular contents of adjacent chambers can also be encouraged through selection of membrane materials. For instance, materials used to form the membrane 23 can include those that can discourage anchorage of cells onto the membrane 23. By way of example, porous membrane 23 can be a polycarbonate membrane. Attachment of cells to membrane 23 can be discouraged to prevent physical contact between cells held in adjacent culture chambers as well as to prevent interference with flow between the adjacent chambers. Interference of flow could, for example, interfere with biochemical communication between the adjacent culture chambers.

In another embodiment the cells contained in one culture chamber 10 can be maintained at a distance from the membrane 23 to discourage physical contact between cells held in adjacent culture chambers. For instance retaining mesh 14 can be located between a cell construct held in a culture chamber 10 and a membrane 23 located between two adjacent chambers. The width of the retaining mesh 14 can prevent contact of the cells with the membrane 23. Optionally, the retaining mesh 14 can be at a distance from the membrane 23, providing additional separation between the membrane 23 and cells held in the culture chamber 10. In another embodiment, a continuous scaffold can be located in a culture chamber 10 at a distance from the membrane 23 so as to discourage physical contact between the cells held in the culture chamber and the membrane 23. While a preferred distance between the membrane 23 and cells held in the chamber can vary depending upon the specific characteristics of the system as well as the cells to be cultured in the system, in general, the distance between the two can be at least about 100 microns.

Each culture chamber of a system can include the capability for independent flow control through the chamber. For example each individual culture chamber 10 can include an inlet 8 and an outlet 9 through which medium can flow and that can be in fluid communication with an individual pressure chamber 3 and downstream chamber 7 as illustrated in FIG. 1. For instance, the inlet and outlet can be connected to tubing via quick-disconnect luers and stopcock valves, but this particular arrangement is not a requirement, and any suitable connection system as is generally known in the art can be utilized. For example, in another embodiment, the connection can be an integral portion of a single formed module 12.

In another embodiment, a co-culture bioreactor system can include a single differential pressure control for two or more of the culture chambers included in the system. For instance, inlet lines to each culture chamber can commence from a single feed source or different feed sources (i.e., containers) held within a single pressure chamber. According to such an embodiment, flow characteristics of medium through each chamber can be essentially identical to one another.

The good flow characteristics possible through utilization of the disclosed differential pressure flow systems can provide for good transport of nutrients to and waste from a developing cell culture, and thus can encourage not only healthy growth and development of the individual cells throughout the culture, but can also encourage development of a unified three-dimensional cellular culture within a culture chamber.

Although only a few exemplary embodiments have been described in detail above, those skilled in the art will readily appreciate that many modifications are possible in the exemplary embodiments without materially departing from the novel teachings and advantages of this disclosure. Accordingly, all such modifications are intended to be included within the scope of this disclosure that is defined in the following claims and all equivalents thereto. Further, it is recognized that many embodiments may be conceived that do not achieve all of the advantages of some embodiments, yet the absence of a particular advantage shall not be construed to necessarily mean that such an embodiment is outside the scope of the present disclosure.

What is claimed is:

1. A biological culture system comprising:
a first pressure chamber;
a first culture chamber located downstream of the first pressure chamber and in fluid communication with the first pressure chamber;
a fluid receptacle located downstream of the first culture chamber and in fluid communication with the first culture chamber; and
a differential pressure control system, wherein fluid flow from the first pressure chamber to the first culture chamber and to the fluid receptacle is controlled via a differential pressure gradient established between the first pressure chamber and the fluid receptacle.

2. The biological culture system of claim 1, wherein the fluid receptacle is a second pressure chamber.

3. The biological culture system of claim 1, further comprising a pliable container that is removably cooperable within the first pressure chamber.

4. The biological culture system of claim 1, further comprising a pliable container that is removably cooperable within the fluid receptacle.

5. The biological culture system of claim 1, further comprising a second culture chamber in biochemical communication with the first culture chamber.

6. The biological culture system of claim 5, wherein the second culture chamber is in fluid communication with the first pressure chamber, wherein fluid flow to the second culture chamber is controlled via the pressure gradient established between the first pressure chamber and the fluid receptacle.

7. The biological culture system of claim 5, wherein the second culture chamber is in fluid communication with a third pressure chamber that is upstream of the second culture chamber, wherein fluid flow to the second culture chamber is controlled via a pressure gradient that is established between the third pressure chamber and a site downstream of the second culture chamber.

8. The biological culture system of claim 1, further comprising two or more additional culture chambers.

9. The biological culture system of claim 1, further comprising a biomaterial scaffold within the first culture chamber.

10. A process for culturing a biological sample comprising:
locating a biological sample in a first culture chamber, wherein said first culture chamber is downstream and in fluid communication with a first pressure chamber and said first culture chamber is upstream of a fluid receptacle;
establishing a differential pressure gradient between the first pressure chamber and the fluid receptacle; and
initiating fluid flow from the first pressure chamber to the first culture chamber.

11. The process according to claim 10, further comprising establishing a pulsatile differential pressure gradient between the first pressure chamber and the fluid receptacle such that the fluid flow from the first pressure chamber to the first culture chamber is a pulsatile flow.

12. The process according to claim 10, further comprising decreasing the differential pressure gradient between the first pressure chamber and the fluid receptacle such that the first culture chamber is held in a state of hydrostatic compression.

13. The process according to claim 10, further comprising decreasing the differential pressure gradient between the first pressure chamber and the fluid receptacle such that the first culture chamber is held in a state of hydrostatic vacuum.

14. The process according to claim 10, further comprising locating a second culture chamber in biochemical communication with the first culture chamber.

15. The process according to claim 14, wherein the second culture chamber is in fluid communication with a second pressure chamber located upstream of the second culture chamber.

16. The process according to claim 10, further comprising locating two or more culture chambers in biochemical communication with one another and with the first culture chamber.

17. The process according to claim 10, wherein the fluid comprises one or more biologically active agents.

18. The process according to claim 10, wherein the culture chamber contains one or more living cell types.

19. The process according to claim 10, wherein the culture chamber contains an engineered tissue construct.

20. The process according to claim 10, wherein the first pressure chamber contains a pressurized gas, the process further comprising controlling the concentration of a gaseous component of the fluid through alteration of the content of the gas held in the first pressure chamber.
